(19) United States
(12) Patent Application Publication
(10) Pub. No.: US 2014/0012280 A1
(43) Pub. Date: Jan. 9, 2014

(54) INTUBATION PROBE

(71) Applicant: FRANCE CHIRURGIE INSTRUMENTATION SAS (FCI), Paris (FR)

(72) Inventor: Stéphane URION, Cromary (FR)

(21) Appl. No.: 13/935,612

(22) Filed: Jul. 5, 2013

(30) Foreign Application Priority Data
Jul. 6, 2012 (FR) 12 01918

(51) Int. Cl. A61F 9/007

(52) U.S. Cl. CPC A61F 9/00772
USPC 606/108

(57) ABSTRACT

An intubation assembly includes an intubation probe adapted to be positioned in a tear duct. The probe includes a flexible tube and a flexible wire has an end which passes through an opening in the tube. The wire is used to pull the probe into the tear duct. The transition between the wire and the tube is continuous, that is, not having any sharp edge which could become stuck in the tear duct wall when the probe is pulled by the wire during displacement from the tear duct.

### TECHNICAL FIELD

The present invention relates to an intubation system, in particular a monocanalicular or bicanalicular system, known as a Ritleng system, which comprises an intubation cannula, a guide wire and a probe, in particular in the form of a tube, in particular made of silicone, the latter being designed for insertion into the tear duct. The present invention also relates to an intubation assembly consisting of a tube probe made of a flexible material and a wire made of a flexible material and connected to the tube probe, as well as a tube probe of this kind.

### BACKGROUND ART

The objective is to introduce the probe in the form of a silicone tube into tear ducts. To achieve this, first of all the tear ducts of the patient are catheterised by means of a cannula open at one end and closed at the opposite end and comprising a slot extending along a generator from the open end towards the closed end, up to an opening formed in the lateral wall of the cannula, said opening having a greater width dimension (measured perpendicular to the longitudinal axis of the cannula) than the slot. Then a flexible wire is connected to the silicone tube to form an intubation assembly and the wire is introduced inside the cannula and is pushed out of the latter through said opening into the nasal cavity. The wire, usually made of prolene, is then retrieved from the nasal cavity and the cannula is removed from the tear ducts by sliding it over the wire and disconnecting it at a narrowed section of the latter by removing said narrowed section through the slot formed over the length of the cannula. Once the cannula has been removed, the wire made of prolene is pulled from the side of the nasal cavity to insert the tube probe made of silicone, joined to the other end of the wire, into the tear ducts. A Ritleng system of this kind is described in French patent FR 2704749.

Although this Ritleng system represented a clear advancement with regard to the introduction of the silicone tube probe into tear ducts, in particular compared to systems such as those described in U.S. Pat. No. 6,117,116 which have a complicated structure and cause bleeding during the insertion of the tube probe into the tear duct, it is desirable to improve the insertion further, in particular make it even less traumatic for the tear ducts and improve the success rate of inserting the silicone tube probe into the tear ducts in a more satisfactory manner.

### DISCLOSURE OF THE INVENTION

The present invention aims to overcome the disadvantages of the prior art by proposing a Ritleng type intubation assembly designed to be inserted into tear ducts by means of a guide wire joined to the probe in order to pull it into position in the tear duct, in which the insertion of the latter is less traumatic for the patient and has a greater success rate of insertion than the prior art.

An intubation assembly according to the invention is defined in claim 1, with refinements being defined in the subordinate claims 2 to 9.

Preferably, the thickness of the tube increases from the opening of the tube along the bevelled section then is constant thereafter.

By providing a bevelled form for the part in the form of a tube of the probe which follows the wire joined to the probe, it is ensured that at the passage between the wire and the silicone tube the transition is more resilient, and consequently when the probe is pulled by the wire to allow it to pass into the tear ducts the tube, because of the bevelled form, penetrates more easily and follows the guide wire more easily, in particular without the edge of the tube turning over on itself, which is less traumatic for the walls of the tear duct. According to the invention and contrary to the probes of the prior art (see for example those described in FR 2704749 or U.S. Pat. No. 6,117,116), the exterior surface of the tube probe, that is the surface of the probe designed to come into contact with the internal wall or walls of the tear duct by sliding against the latter during the insertion of the probe into the duct, does not have a sharp edge which could jam or get stuck in the wall, which would cause the folding or turning over of the probe on itself or lesions or traumas on the wall of the duct.

Furthermore, the structure of the assembly is simple, formed simply by the wire and the probe, without requiring an additional connecting piece, which on the one hand is a potential complication for the manufacturing and on the other hand affects the rigidity of the assembly at the join, which makes the assembly less easy to manipulate by the surgeon during the insertion.

The present invention also relates to an intubation probe designed to be connected to a guide wire to form an assembly according to the invention, as well as to an intubation system comprising an intubation assembly according to the invention and an intubation cannula, the cannula being inserted prior to the introduction of the Ritleng type intubation assembly into the tear duct.

### BRIEF DESCRIPTION OF THE DRAWINGS

By way of example, an embodiment of the invention will now described with reference to the drawings in which:

FIG. 1 is a view from above of a cannula of an intubation system according to the invention;

FIG. 2 shows an intubation assembly forming a Ritleng probe according to the invention, which shows the connection between the silicone tube and the guide wire;

FIG. 3 is a schematic view describing the method of inserting the silicone tube into the tear duct;

FIG. 4 shows another embodiment of a cannula of an intubation system according to the invention, shown in longitudinal cross section; and

FIG. 5 is view from above of the cannula of FIG. 4.

### PREFERRED EMBODIMENT FOR CARRYING OUT THE INVENTION

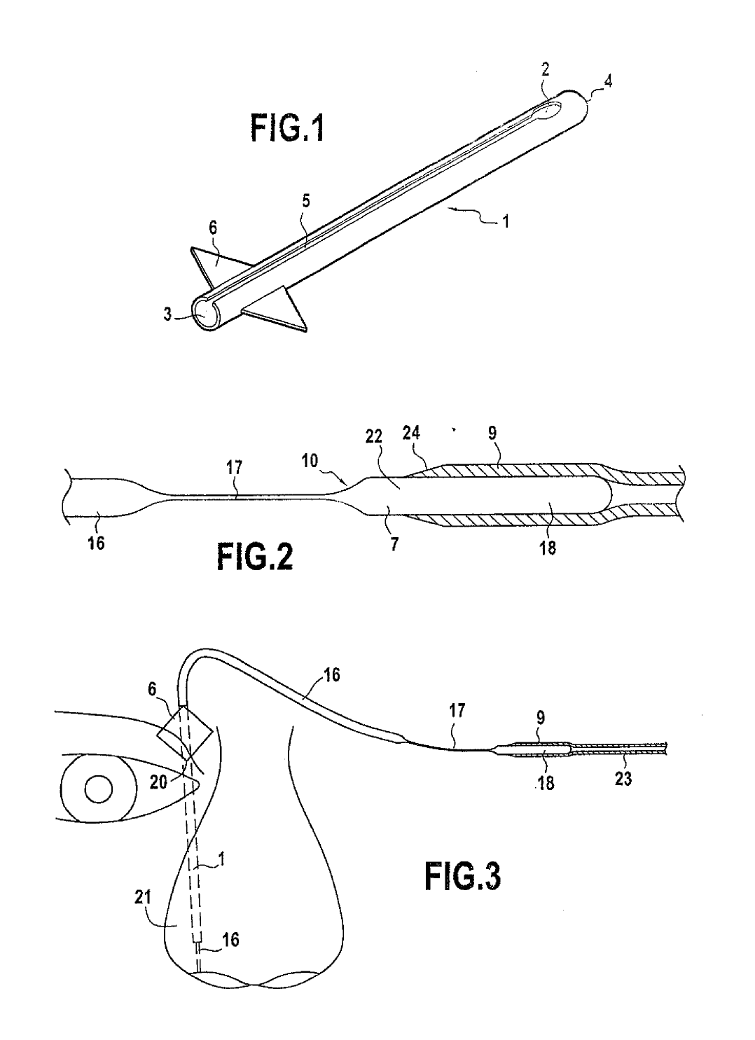

FIG. 1 shows a cannula of a system according to the invention. Said cannula 1 is formed by a hollow circular cylindrical body made from a semi-rigid material, for example stainless steel or a thermoplastic material, in particular PEEK or polyarylamide, open at a proximal end 3 and closed at the opposite distal end 4.

The cannula 1 comprises a longitudinal slot 5 which extends along a generator of the cylinder, from the open proximal opening 3 in the direction of the closed end 4 up to an opening 2 with a larger width (measured perpendicular to the longitudinal axis of the cylinder, which is parallel to the longitudinal slot) than that of slot 5.

The opening 2, viewed from above as in FIG. 1, has an elliptical form, the small diameter of which is perpendicular to the longitudinal axis and the large diameter of which is parallel to the axis of the slot. The small diameter is greater than the thickness of the gap of the slot.

Gripping fins 6 project laterally from the cannula, adjacent to the proximal opening 3.

FIG. 2 shows in part an intubation assembly 10 known as a Ritleng system. This Ritleng type intubation assembly 10 is formed by a wire 7, in particular made of prolene or another biocompatible and similarly resilient material, and a tube probe 9 made of silicone.

The wire has three sections, namely an end section 16 with a large diameter followed by an intermediate section 17 with a smaller diameter, the latter followed by an opposite end section 18 with a large diameter. The diameter of the section 16 with a large diameter is such that it can pass into the tube and into the opening 2 but not into the slot 5. The diameter of the intermediate section 17 with a smaller diameter is such that it can pass into the slot 5.

The probe 9 is formed by a hollow circular cylinder with a wall width of preferably between 0.15 mm and 0.5 mm. It is made in particular of silicone or another similar material.

The opposite end section 18 with a large diameter is inserted into the silicone tube 9 for their mutual connection. Said connection can be performed by inserting with force or by means of a tight fit. It is also possible to connect them by adhesion or any other similar method, for example ultrasonic welding.

The insertion of the tube probe 9 into the tear duct is performed in the following manner:

First of all the cannula 1 is inserted into the tear duct by penetrating through the exterior entry point 20 from the side of the eye up to the exit 21 opening into the nasal cavity.

Once the cannula has been inserted section 16 is slid along until the end of the section comes out of the cannula through the opening 2. The surgeon then takes hold of the end coming out of section 16 and by taking hold of it, for example with a suitable pair of tweezers (not shown), he pulls the cannula 1 by the fins out of the tear duct. To insert the tube probe 9 into the tear duct, the opposite end section 18 with a large diameter is inserted into the cannula and by taking hold of the latter by the fins 4, the assembly (cannula and wire) is inserted into the tear duct from the exterior from the side of the eye until one end of the wire passing through the opening 2 of the cannula comes out through the inside of the nostrils. Then the cannula 1 is removed by pulling on the fins 4. The cannula slides along the wire until it reaches the intermediate section 17 which can then be passed through the slot 5 of the cannula to release the latter from its cooperation with the wire. Once the cannula 1 has been removed, the wire is pulled by its end coming from the tear duct from the side of the nostril until the silicone tube 9, joined to section 18 of the wire, is inserted into the tear duct. Once the silicone tube 9 has been inserted into the tear duct, the guide wire which hangs from the exterior of the nostrils is cut.

The probe 9 is formed here by a tube open on both sides. From the side of the opening 22 through which section 16 is introduced into the tube 9 for the mutual connection thereof, the lateral wall 23 defining the tube has a thickness which varies such that a section (24) of the edge of the tube 9 has a bevelled form.

During the installation of the silicone tube 9 by pulling the guide wire, the fact that the leading edge of the guiding tube 9 has a bevelled form makes it possible to assist with the installation of the silicone tube 9 such that, unlike the prior art, there is no leading edge with a sharp edge which knocks against the wall of the tear duct, thus avoiding damage to this wall of the tear duct and/or the edge of the tube 9 does not turn over which may cause the disconnection of section 16 and the tube 9 before reaching the final desired position of the latter in the tear duct and therefore there is no poor positioning of the silicone tube 9 making it necessary to locate it, then restart the whole operation with a new silicone tube. Thus, according to the invention, by means of the bevelling or chamfering the insertion is less traumatic for the wall of the tear duct and has a greater rate of success.

The exterior surface of the probe 9, that is the surface intended to come into contact with the wall of the tear duct during its insertion into the duct, in particular by sliding against this wall, does not have a sharp edge, said exterior surface having an inclined end section 24. The section 24 extends up to its free end such that when the wire 22 is inserted into the tube, there is no longer a sharp edge between the wire and the section 24 at the free end of the latter, the transition between the part of the wire 22 on the exterior of the tube 9 and the second being resilient, without any roughness or sharp edge which could get stuck or jammed in the wall of the duct.

During the insertion, the exterior surfaces of the wire and the probe adjacent to the opening 22 of the interface between the wire and the probe form the exterior of the assembly and come into direct contact with the wall of the tear duct.

The flexibility (or rigidity, reverse of flexibility) of the assembly is such that at any point over its length, and in particular at the connection point of the wire to probe, the assembly can be deformed, for example can curve to take the form of any curve or can fold to form an angle and take on the form of a crossing of ducts. In particular, at any point the assembly is sufficiently flexible to take on a curved form and maintain it without returning to a straight position in the manner of a wire.

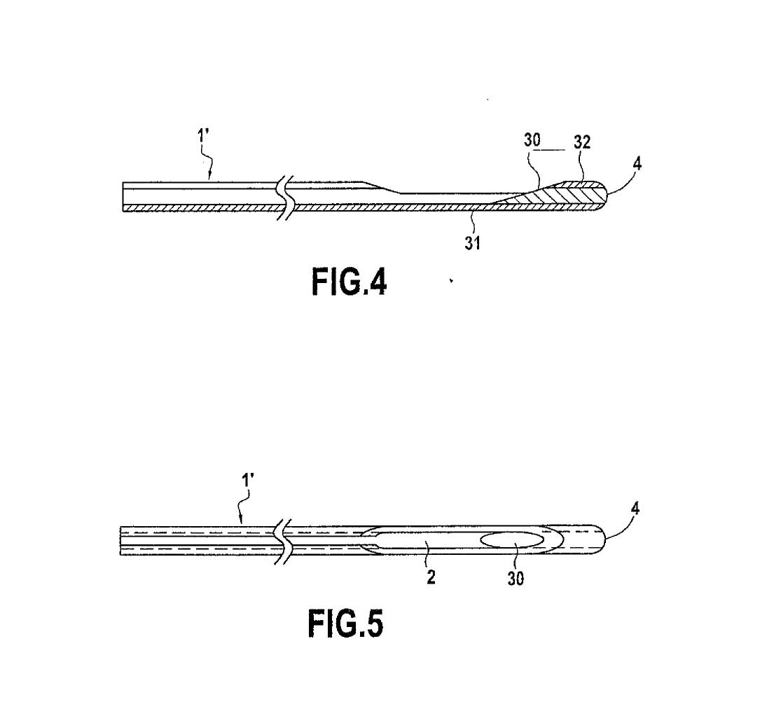

FIGS. 4 and 5 show a cannula 1′ according to another embodiment which can also be used in the intubation system described above in place of the cannula 1. Identical parts of the two cannulas 1 and 1′ are represented by the same reference numbers.

Beyond the opening 2 and up to the opposite distal end 4, the cannula is solid such that the guide wire, during its insertion into the cannula, cannot penetrate into the cannula beyond the opening 2, abutting against the inner surface 30 of the base of the cannula. Said inner surface 30 is bevelled, being inclined by about 18° relative to the longitudinal axis of the cannula.

The inner surface 30 forming the base of the cannula is planar. In longitudinal cross section, as shown in FIG. 4, it has the form of a straight line which extends from a lower point 31 of the inner side wall of the cannula from the side opposite the slot up to an upper point 32 of the side wall of the cannula from the side of the slot, the upper point 32 being closer to the distal end 4 of the cannula than point 31. In place of a planar form it is also possible however to have a slightly curved, concave or convex form. The function of this surface 30 forming the inner base of the cannula is to guide the end of the guide wire to make it exit through the opening 2 during the insertion stage of the wire into the cannula, and thus prevent it from penetrating into an area of the cannula beyond the opening 2, between the latter and the distal end, to prevent, as in the prior art, the wire from getting stuck and requiring the surgeon to retrieve the stuck wire from the space beyond the opening.

According to another embodiment, which is not shown, it is possible not to fill the space beyond the opening 2 but to close it with a wall which then forms an abutting and guiding surface for the guide wire with the same function as the surface 30.

What is claimed is:

1. Intubation assembly comprising:
an intubation probe designed to be positioned in the tear duct, comprising at least one part in the form of a tube (9) made from a flexible, biocompatible material; and
a wire (7), made from a flexible material and joined to the probe, by means of which the probe is pulled to be moved into position in the tear duct;
in which an end section (16) of the wire extends inside the tube probe passing through an opening (22) of the latter, characterised in that the transition between the wire and the tube probe is formed to be continuous, that is without any sharp edge which could get stuck in the tear duct wall during the displacement of the probe pulled by the wire.

2. Assembly according to claim 1, characterised in that from the opening (22) the tube probe has a bevelled section, the exterior diameter of the tube increasing from the opening (22) by an amount substantially equal to the diameter of the wire.

3. Assembly according to claim 2, characterised in that after the bevelled section the tube probe continues in the form of a cylindrical tube.

4. Assembly according to claim 1, characterised in that the thickness of the tube is substantially zero at the opening (22), then increases along the bevelled section such that when the wire is introduced into the tube the transition between the wire and the tube is flexible, substantially in a continuous manner, without any roughness or sharp edges.

5. Assembly according to claim 1, characterised in that the wire is joined to the tube by means of the tight fit of the latter around the wire.

6. Assembly according to claim 1, characterised in that the wire is connected to the tube by adhesion.

7. Assembly according to claim 1, characterised in that the assembly is only formed by the wire and the tube probe, in particular without a connecting piece at the join between the wire and the probe.

8. Assembly according to claim 1, characterised in that the outer surface of the assembly at the opening (22) is not covered.

9. Assembly according to claim 1, characterised in that at every point the assembly is sufficiently flexible to take on a curved form and maintain it without returning to a straight position, in the manner of a wire.

10. Intubation system comprising an intubation assembly according to claim 1 and an intubation cannula, the cannula being designed to be inserted into the tear duct prior to the introduction of the intubation assembly.

11. Tube probe designed to form part of an assembly as defined in claim 1.
